(19) United States
(12) Patent Application Publication
(10) Pub. No.: US 2023/0125487 A1
(43) Pub. Date: Apr. 27, 2023

(54) COMPOSITION FOR TREATING OR PREVENTING INFLAMMATORY BOWEL DISEASE

(71) Applicant: FUGENBIO CO., LTD., Seoul (KR)

(72) Inventors: Jin-Mo KU, Suwon-si, Gyeonggi-do (KR); Sung Kyoon YOON, Suwon-si, Gyeonggi-do (KR); Dawoon SONG, Suwon-si, Gyeonggi-do (KR); Ji-Man JUNG, Incheon (KR); Eugene JEONG, Seoul (KR)

(73) Assignee: FUGENBIO CO., LTD., Seoul (KR)

(21) Appl. No.: 17/626,639

(22) PCT Filed: Jul. 13, 2020
(86) PCT No.: PCT/KR2020/009156

§ 371 Date: Jan. 17, 2023

(30) Foreign Application Priority Data
Jul. 12, 2019 (KR) 10-2019-0084738

(51) Int. Cl. C07C 49/223

(52) U.S. Cl. CPC C07C 49/223

(57) ABSTRACT

The present invention relates to a compound or a salt thereof which can be usefully used for the treatment, improvement, or prevention of inflammatory bowel disease. In addition, it relates to a pharmaceutical composition for treating or preventing inflammatory bowel disease comprising the compound or a pharmaceutically acceptable salt thereof. In addition, it relates to a food composition for improving or preventing inflammatory bowel disease comprising the compound or a sitologically acceptable salt thereof.

### TECHNICAL FIELD

The present invention relates to a compound or a salt thereof which can be usefully used for the treatment, improvement, or prevention of inflammatory bowel disease. In addition, it relates to a pharmaceutical composition for treating or preventing inflammatory bowel disease comprising the compound or a pharmaceutically acceptable salt thereof. In addition, it relates to a food composition for improving or preventing inflammatory bowel disease comprising the compound or a sitologically acceptable salt thereof.

### BACKGROUND ART

Patients suffering from inflammatory bowel disease cannot lead a normal daily life due to extreme pain and spend a lot of time and money for treatment. In addition, inflammatory bowel disease is difficult to cure in a short period of time and often progresses chronically. Such inflammatory bowel diseases include, for example, ulcerative colitis, Crohn's disease, and the like.

Ulcerative colitis and Crohn's disease have similar symptoms, such as diarrhea, severe abdominal pain, nausea, fever, anorexia, weight loss, and fatigue. While the ulcerative colitis develops thinly on the intestinal surface of the large intestine, Crohn's disease occurs in the entire digestive tract from the mouth to the anus, mainly in the small and large intestines, and is distributed to the deep part of the mucous membrane, sometimes resulting in perforation of the intestinal wall.

The Crohn's disease is a disease commonly occurring in developed countries in the West, and it was a very rare disease in the East. However, the incidence of Crohn's disease in East Asia including South Korea has been rapidly increasing since the 1980s, and the number of patients with Crohn's disease in 2015 increased by 31.7% compared to 2011 according to the Korea Health Insurance Review and Assessment Service. Although the cause of Crohn's disease has not been clearly identified, it is presumed to be caused by the interaction of various factors such as genetics, immunity, and environmental factors. There is also a report that a person with a genetically high risk of Crohn's disease is exposed to specific environmental factors, and then a continuous immune response against normal intestinal bacteria occurs in the intestine, thereby triggering a chronic inflammatory disease and causing Crohn's disease.

According to the treatment guidelines for Crohn's disease, anti-inflammatory drugs, steroids, immunosuppressants, or biological agents are prescribed depending on the severity of symptoms.

Drugs primarily used to treat the Crohn's disease include 5-aminosalicylic acid (5-ASA), and sulfasalazine and mesalazine are typical examples. The sulfasalazine is a drug that has been basically used for decades in the medical treatment of Crohn's disease, and is a drug that is first administered for induction and maintenance therapy in patients with mild and severe colorectal Crohn's disease. Although the therapeutic effect is proportional to the dose, there are disadvantages in that side effects such as headache, nausea, heartburn, dizziness, anemia, skin rash, and photosensitivity may occur when high doses are administered. The mesalazine is a drug developed to solve the side effects of sulfasalazine, has a low incidence of side effects of sulfasalazine, and can be safely used during pregnancy. However, it has been reported that other side effects such as diarrhea, indigestion, and rash may still occur.

The steroid is a drug that suppresses the immune response by variously affecting the body's immune and inflammatory responses, and is mainly used when 5-ASA is ineffective and is used only for induction therapy. However, when it is used for a long time, there is a problem that there may be restrictions on infection, trauma, surgery, and the like.

The immunosuppressant is a drug that suppresses the immune system response in the body so as not to cause continuous inflammation, and is used for steroid dependent patients who have not obtained an effect from 5-ASA or steroid preparations or who are concerned about steroid side effects. However, there are problems in that side effects such as nausea, pancreatitis, leukocyte reduction, decreased resistance to infection, skin rash, fever, and arthralgia may occur.

The biological agent has been recently studied as a drug that selectively inhibits the pro-inflammatory cytokine TNFα. It has an advantage of being rapidly absorbed into the body as an injection, but has a problem in that it does not respond in ⅓ of all patients and may cause a decrease in treatment response due to antibody formation. In addition, there are disadvantages that it is difficult for the patient to easily administer the injection and that the price is high.

In addition to drugs for treating Crohn's disease, surgical treatment and the like are performed. The surgical treatment is performed by cutting out a part of the inflamed intestine and then connecting both healthy intestines. However, these surgeries do not cure the disease and have a high risk of recurrence.

Although Crohn's disease patients cannot live a normal life due to extreme pain and spend a lot of time and money for treatment, most of the Crohn's disease treatments developed so far are synthetic materials, except for biological agents, and have serious side effects. In addition, in most cases, remission is not resolved or cannot be maintained even after treatment with conventional therapeutic agents. Therefore, there is a high need to develop a novel therapeutic agent capable of exhibiting efficacy with the use of a single agent having higher efficacy than conventional therapeutic agents without side effects.

### Technical Problem

The inventors recognized the problems of the prior art and repeated research through numerous trials and errors in order to find a compound having excellent therapeutic or preventive effects on patients with inflammatory bowel disease. As a result, we have developed a compound that can have excellent treatment, improvement, and preventive effects on patients with inflammatory bowel disease through anti-inflammatory, immunosuppressive, and TNFα inhibitory effects, and have completed the present invention.

### Technical Solution

A compound according to the present invention for achieving the object of the present invention is represented by the Formula 1:

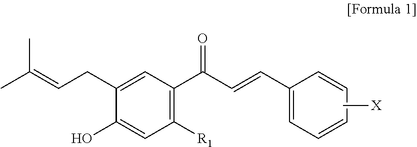

wherein,

R₁ is H, OH or halogen; and

X is one to five substituent group independently selected from H, OH, alkoxy and halogen.

As used herein, the term “halogen” means fluorine, chlorine, bromine, or iodine.

As used herein, the term “alkoxy” refers to O-alkyl, wherein alkyl is intended to include both linear and branched forms.

The number of substituent X may be 1, or 2 or more, for example, 2, 3, 4 or 5. When there are two or more substituents X, each may be the same or different from each other, and may be ortho, meta, or para positions with each other.

Preferably, broussochalcone A is not included in the compound of Formula 1. Brausochalcon A is referred to as A1 in the experiments described below, and has the formula:

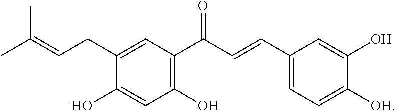 [SMILES: CC(C)=CCc1cc(C(=O)/C=C/c2ccc(O)c(O)c2)c(O)cc1O]

Since it has been already disclosed that the broussochalcone A inhibits the excessive free radicals and NO formation (refer to CHENG, ZHI-JIAO, et al., Biochemical Pharmacology, 2001, Vol. 61, pages 939-946), it was used as a positive control group to compare the effect of the compound according to the present invention.

The compound of Formula 1 may have an asymmetric carbon center, and thus it may exist as an R or S isomer, a racemate, a diastereomer, or a mixture thereof, all of which are included in the scope of the present invention.

The term “pharmaceutically or sitologically acceptable salt” used herein refers to a compound that can be prepared by a conventional method in the art, for example, hydrochloric acid, hydrogen bromide, sulfuric acid, sodium hydrogen sulfate, phosphoric acid, carbonic acid, etc. salts with inorganic acids of formic acid, acetic acid, oxalic acid, benzoic acid, citric acid, tartaric acid, gluconic acid, gestisic acid, fumaric acid, lactobionic acid, salicylic acid; or use organic acids such as acetylsalicylic acid (aspirin) to form pharmaceutically or sitologically acceptable acid salts thereof; or react with an alkali metal ion such as sodium or potassium to form a metal salt thereof; or react with an ammonium ion to form another pharmaceutically or sitologically acceptable salt, but is not limited thereto.

The present invention provides a composition for treating or preventing inflammatory bowel disease, comprising the compound represented by Formula 1 or a pharmaceutically acceptable salt thereof as an active ingredient.

As used herein, the term “treatment” or “treating” refers to all activities in which symptoms of inflammatory bowel disease are improved or cured by administration of the composition according to the present invention.

As used herein, the term “prevention” or “preventing” refers to all activities in which inflammatory bowel disease is suppressed or delayed by administration of the composition according to the present invention.

The inflammatory bowel disease may include, but is not limited to, ulcerative colitis, Crohn's disease, and the like. It should be understood that the inflammatory bowel disease includes all symptoms, diseases, conditions, etc. which additionally occur as inflammatory bowel disease progresses, occur concurrently with inflammatory bowel disease, or increase the severity of inflammatory bowel disease.

The pharmaceutical composition according to the present invention may contain the compound represented by Formula 1 or a pharmaceutically acceptable salt thereof alone or may additionally contain one or more pharmaceutically acceptable carriers, excipients or diluents.

The pharmaceutically acceptable carrier may include, for example, a carrier for oral administration or a carrier for parenteral administration. The carrier for oral administration may include lactose, starch, cellulose derivatives, magnesium stearate, stearic acid, and the like. In addition, the carrier for parenteral administration may include water, suitable oil, saline, aqueous glucose, glycol, and the like, and may further include a stabilizer and a preservative. Suitable stabilizers include antioxidants such as sodium bisulfite, sodium sulfite or ascorbic acid. Suitable preservatives include benzalkonium chloride, methyl- or propyl-paraben and chlorobutanol. As other pharmaceutically acceptable carriers, reference may be made to those described in the literature: Remington Pharmaceutical Sciences, 19th ed., Mack Publishing Company, Easton, Pa., 1995.

The pharmaceutical composition of the present invention may be administered to mammals including humans by any method. For example, it can be administered orally or parenterally. Parenteral administration methods include, but are not limited to, intravenous, intramuscular, intraarterial, intramedullary, intrathecal, intracardiac, transdermal, subcutaneous, intraperitoneal, intranasal, enteral, topical, sublingual, or rectal administration. For example, the pharmaceutical composition of the present invention may be prepared as an injectable formulation and administered by lightly pricking the skin with a 30 gauge thin injection needle or by directly applying the composition to the skin.

The pharmaceutical composition of the present invention may be formulated into a preparation for oral administration or parenteral administration according to the administration route as described above.

For the preparations for oral administration, the composition of the present invention may be formulated into powders, granules, tablets, pills, dragees, capsules, solutions, gels, syrups, slurries, suspensions, etc. using a method known in the art. For example, preparations for oral use may be obtained by combining the active ingredient with a solid excipient, milling it, adding suitable auxiliaries, and processing into a mixture of granules to obtain tablets or dragees. Examples of suitable excipients may include sugars including lactose, dextrose, sucrose, sorbitol, mannitol, xylitol, erythritol and maltitol, starches including corn starch, wheat starch, rice starch and potato starch, cellulose including methyl cellulose, sodium carboxymethylcellulose and hydroxypropylmethyl-cellulose, fillers such as gelatin, polyvinylpyrrolidone, and the like. In addition, cross-linked polyvinylpyrrolidone, agar, alginic acid or sodium alginate may be added as a disintegrant, if desired. Furthermore, the composition of the present invention may further comprise an anti-agglomerating agent, a lubricant, a wetting agent, a flavoring agent, an emulsifying agent, and a preservative.

For the preparations for parenteral administration, the composition of the present invention may be formulated in the form of injections, creams, lotions, external ointments, oils, moisturizers, gels, aerosols, and nasal inhalants by methods known in the art. These formulations are described in Remington's Pharmaceutical Science, 15th Edition, 1975. Mack Publishing Company, Easton, Pa. 18042, Chapter 87: Blaug, Seymour, which is generally known in the field of all pharmaceutical chemistry.

The total effective amount of the pharmaceutical composition of the present invention may be administered to the patient in a single dose, or by a fractionated treatment protocol in which multiple doses are administered over a long period of time. The pharmaceutical composition of the present invention may vary the content of the active ingredient depending on the symptoms of the disease. Preferably, a total dose of the composition of the present invention may be from about 0.01 μg to 1,000 mg, more preferably from 0.1 μg to 100 mg per kg of patient body weight per day. However, a person skilled in the art may determine an appropriate effective dosage of the pharmaceutical composition of the present invention considering various factors including not only the route of administration and the number of treatments, but also the patient's age, weight, health condition, gender, severity of disease, diet and excretion rate, etc. The pharmaceutical composition according to the present invention is not particularly limited in its formulation, administration route and administration method as long as it exhibits the effects of the present invention.

In addition, the pharmaceutical composition of the present invention may be administered as an individual therapeutic agent or in combination with other therapeutic agents. When administered in combination with other therapeutic agents, they may be administered simultaneously, separately or sequentially. The other therapeutic agents may be a substance already known to have an effect of treating or improving inflammatory bowel disease. They include all surgical procedures other than drug therapy, and surgery.

When the pharmaceutical composition of the present invention is administered in combination with other therapeutic agents, they may be separately formulated in separate containers or formulated together in the same container.

In another aspect of the present invention, a method for treating or preventing inflammatory bowel disease comprising administering the composition of the present invention to a subject is provided.

In the method for treating or preventing inflammatory bowel disease according to the present invention, each term has the same meaning as described above in the pharmaceutical composition for treating or preventing inflammatory bowel disease, unless otherwise specified.

The term “subject” includes any human or non-human animal. The term “non-human animal” may include vertebrates such as non-human primates, sheep, dogs, and rodents such as mice, rats and guinea pigs. The subject may preferably be a human. The term “subject” may be used interchangeably with “individual” and “patient” herein.

In the method for treating or preventing inflammatory bowel disease according to the present invention, the composition of the present invention may be administered to a subject simultaneously, sequentially, or separately with other therapeutic agents. The “simultaneous” administration means that the composition of the present invention and other therapeutic agents are administered at one time through the same administration method. The “sequential” administration means that the composition of the present invention and other therapeutic agents are administered relatively continuously using separate administration methods, and the minimum time required for administration intervals is allowed. The “separate” administration means that the composition of the present invention and other therapeutic agents are administered at regular time intervals. The administration method of the composition of the present invention and other therapeutic agents can be appropriately determined by those skilled in the art in consideration of the therapeutic efficacy and side effects of patients.

In another aspect of the present invention, it provides a food composition for improving or preventing inflammatory bowel disease, comprising the compound represented by Formula 1 or a sitologically acceptable salt thereof as an active ingredient.

In the food composition according to the present invention, each term has the same meaning as described above in the pharmaceutical composition for the treatment or prevention of inflammatory bowel disease, unless otherwise specified.

As used herein, the term “improvement” or “improving” refers to any activity in which the severity of inflammatory bowel disease or abnormal symptoms is reduced, ameliorated, or the progression is delayed by administration of the composition according to the present invention.

The food may be a health functional food. The term “health functional food” refers to food manufactured and processed in the form of tablets, capsules, powders, granules, liquids and pills using raw materials or ingredients having useful functionality for the human body. Here, “functionality” means obtaining useful effects for health purposes such as adjusting nutrients for the structure and function of the human body or physiological functions.

The food composition according to the present invention may be prepared by a method commonly used in the art, and raw materials and components commonly used in the art may be added during the preparation. In addition, unlike general drugs, it has an advantage that there is no side effect which may occur when taking a drug for a long time, and it has excellent portability. Thus, the food composition of the present invention may be taken as an adjuvant for promoting or improving the therapeutic effect of inflammatory bowel disease.

The amount of compound or salt thereof comprised as an active ingredient in the food composition according to the present invention may be appropriately determined depending on the purpose of use such as prevention, improvement, or therapeutic treatment. In general, when preparing food, the compound or salt thereof of the present invention may be contained in an amount of 0.001 to 20% by weight, 0.001 to 15% by weight, or 0.001 to 10% by weight in the composition. In case of health drinks, 0.01 to 2 g, specifically 0.02 to 2 g, and more specifically 0.3 to 1 g may be added based on 100 mL. However, for a long-term intake for the purpose of health and hygiene or health control, the amount may be less than the above range.

In the process of preparing the food composition, the content of the compound or salt thereof according to the present invention added to the food composition may be appropriately increased or decreased as necessary.

The food composition of the present invention may further comprise other ingredients in addition to the compound or salt thereof to enhance efficacies.

The food composition may be any one formulation selected from the group consisting of pills, tablets, granules, powders, capsules, and liquid solutions.

In addition, the type of food is not particularly limited. Examples of foods to which the composition can be added include meat, sausage, bread, chocolate, candy, snacks, confectionery, pizza, ramen, other noodles, gum, dairy products including ice cream, various soups, beverages, tea, drinks, alcoholic beverages, vitamin complexes, and the like, and includes all foods in a conventional sense.

The food composition of the present invention may comprise various flavoring agents or natural carbohydrates as additional components, like conventional foods. The natural carbohydrates include monosaccharides such as glucose and fructose, disaccharides such as maltose and sucrose, polysaccharides such as dextrin and cyclodextrin, and sugar alcohols such as xylitol, sorbitol, and erythritol. As the sweetener, natural sweeteners such as thaumatin and stevia extract, or synthetic sweeteners such as saccharin and aspartame may be used.

In case the food composition of the present invention is a beverage composition, there is no particular limitation on the liquid component, except that the compound or salt thereof is contained in the indicated ratio as an essential component, and various flavoring agents or natural carbohydrates may be contained as in conventional beverages.

The compound or salt thereof according to the present invention provides significant treatment, prevention, and improvement effects on inflammatory bowel disease.

In particular, it can have excellent treatment, improvement, and preventive effects on patients with inflammatory bowel disease through anti-inflammatory, immunosuppressive, and TNFα inhibitory effects. In addition, it has no or almost insignificant side effects such as headache, nausea, heartburn, etc., which are found in conventional therapeutic agents.

Moreover, when it is produced according to the preparation steps described in the Examples to be described below, mass production is possible at low cost. This can reduce the high cost of conventional therapeutic agents. In particular, when a pharmaceutical composition or food composition should be administered for a long period of time due to the nature of inflammatory bowel disease, it can reduce the burden of increasing cost.

Therefore, the composition comprising the compound or salt thereof according to the present invention can be applied as a pharmaceutical composition for preventing or treating inflammatory bowel disease.

In addition, the composition comprising the compound or salt thereof according to the present invention can be applied as a food composition for improving or preventing inflammatory bowel disease.

### BRIEF DESCRIPTION OF FIGURES

The Figures attached to this specification show the average value of the data repeated three times.

FIGS. 1a to 1c are graphs showing changes in the mRNA expression level of the inflammatory cytokine TNFα increased by LPS stimulation in RAW 264.7 macrophages.

FIGS. 2a and 2b are graphs representing the mRNA expression level of the inflammatory cytokine COX2 increased by LPS stimulation in RAW 264.7 macrophages.

FIGS. 3a and 3b are graphs representing the mRNA expression level of the inflammatory cytokine iNOS increased by LPS stimulation in RAW 264.7 macrophages.

FIG. 4 is a graph representing the mRNA expression level of the inflammatory cytokine IL-1α increased by LPS stimulation in RAW 264.7 macrophages.

FIG. 5 is a graph representing the mRNA expression level of the inflammatory cytokine IL-1β increased by LPS stimulation in RAW 264.7 macrophages.

FIG. 6 is a graph representing the mRNA expression level of the inflammatory cytokine MCP1 increased by LPS stimulation in RAW 264.7 macrophages.

FIGS. 7a and 7b are graphs showing changes in NO concentration (μM) increased by LPS stimulation in RAW 264.7 macrophages.

### EXAMPLES

Hereinafter, the present invention will be described in more detail by examples. However, the examples are provided for easier understanding of the present invention, and the contents of the present invention are not limited by the following examples. In addition, those skilled in the art will be able to make various changes and modifications to the present invention within a range that does not impair the spirit of the present invention. Terms not specifically defined herein should be understood to have meanings commonly used in the technical field to which the present invention belongs.

In the following, examples of preparation of compounds of Formula 1 according to the present invention are described together with specific preparation steps and representative examples corresponding thereto. Compounds having different substituents were prepared through similar steps, but not all examples are described here. Referring to the following representative examples, those skilled in the art will be able to easily prepare compounds of Formula 1 having different substituents.

### Preparation Example 1

A compound in which the substituent R₁ is OH in Formula 1 was prepared according to the following steps.

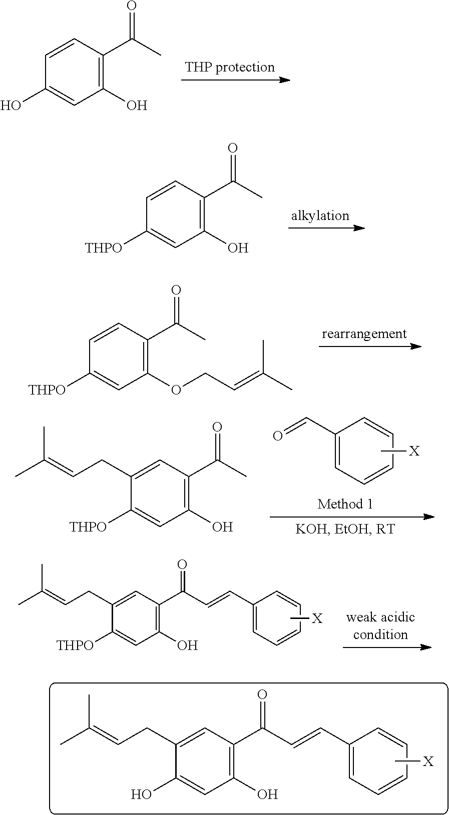

In order to introduce a prenyl group at position 3 of the starting material acetophenone, an alkylation reaction and a rearrangement reaction were performed. After that, when the reaction was performed using a strong base such as KOH or NaOH, the yield was low and it was difficult to separate and purify it if the material comprises a specific hydroxyl group. In order to solve this problem, the inventors protected the —OH group using DHP (dihydropyran) and proceeded with the condensation reaction of acetophenone and various benzaldehydes, and then deprotected the THP group under weakly acidic conditions to obtain the desired final product.

Chemical structures and ¹H NMR, ¹³C NMR, LRMS, HRMS, and m.p values of Compounds A1 to A6 prepared in Preparation Example 1 are shown below, respectively.

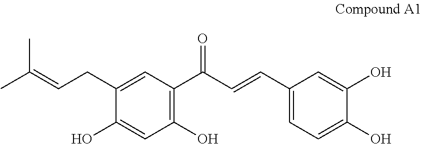 [SMILES: CC(C)=CCc1cc(C(=O)/C=C/c2ccc(O)c(O)c2)c(O)cc1O]

¹H NMR (400 MHz, acetone) δ 13.53 (1H, s), 8.68 (2H, brs), 7.96 (1H, s), 7.77 (1H, d, J=15.2 Hz), 7.68 (1H, d, J=7.2 Hz), 7.68 (1H, d, J=2.0 Hz), 7.21 (1H, dd, J=8.0, 2.0 Hz), 6.92 (1H, d, J=8.4 Hz), 6.41 (1H, s), 5.38-5.33 (1H, m), 3.32 (2H, d, J=7.2 Hz), 1.74 (6H, d, J=8.0 Hz); ¹³C NMR (100 MHz, acetone) δ 192.7, 165.8, 163.4, 149.1, 146.3, 145.2, 132.2, 128.3, 123.9, 123.4, 121.3, 118.5, 116.4, 115.8, 114.3, 103.4, 28.8, 25.9, 17.9; LRMS (ESI) calcd. for C₂₀H₂₁O₅ [M+H]⁺: 341.13, found: 341.10; HRMS (ESI) calcd. for C₂₀H₂₁O₅ [M+H]⁺: 341.1311, found: 341.1395; m.p=192.6° C.

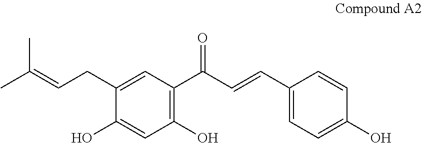 [SMILES: CC(C)=CCc1cc(C(=O)/C=C/c2ccc(O)cc2)c(O)cc1O]

¹H NMR (400 MHz, acetone) δ 13.50 (1H, s), 9.44 (1H, brs), 8.96 (1H, brs), 7.96 (1H, s), 7.83 (1H, d, J=17.2 Hz), 7.76 (1H, s), 7.71 (2H, d, J=8.4 Hz), 6.95 (2H, d, J=11.6 Hz), 6.41 (1H, s), 5.38-5.33 (1H, m), 3.32 (2H, d, J=6.8 Hz), 1.74 (6H, d, J=8.4 Hz); ¹³C NMR (100 MHz, acetone) δ 192.7, 165.9, 160.9, 144.8, 132.3, 132.2, 131.6, 127.6, 123.9, 121.3, 118.5, 118.4, 116.7, 114.3, 103.4, 28.8, 25.8, 17.9; LRMS (ESI) calcd. for C₂₀H₂₁O₄ [M+H]⁺: 325.13, found: 325.10; HRMS (ESI) calcd. for C₂₀H₂₁O₄ [M+H]⁺: 325.1362, found: 325.1443; m.p=170.6° C.

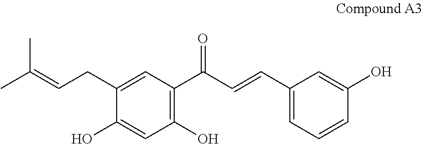 [SMILES: CC(C)=CCc1cc(C(=O)/C=C/c2cccc(O)c2)c(O)cc1O]

¹H NMR (400 MHz, acetone) δ 13.35 (1H, s), 9.55 (1H, brs), 8.59 (1H, brs), 7.99 (1H, s), 7.86 (1H, d, J=15.6 Hz), 7.79 (1H, d, J=15.6 Hz), 7.33-7.25 (3H, m), 6.99-6.94 (1H, m), 6.43 (1H, s), 5.38-5.33 (1H, m), 3.32 (2H, d, J=6.8 Hz), 1.74 (6H, d, J=8.0 Hz); ¹³C NMR (100 MHz, acetone) δ 192.6, 165.9, 158.7, 144.6, 137.3, 132.4, 130.8, 123.8, 121.7, 121.5, 121.0, 118.5, 115.9, 114.2, 103.4, 28.7, 25.8, 17.9; LRMS (ESI) calcd. for C₂₀H₂₁O₄ [M+H]⁺: 325.13, found: 325.10; HRMS (ESI) calcd. for C₂₀H₂₁O₄ [M+H]⁺: 325.1362, found: 325.1447; m.p=196.2° C.

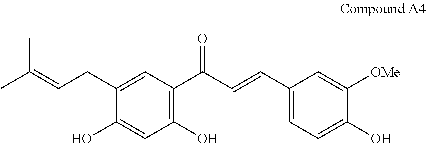 [SMILES: COc1cc(/C=C/C(=O)c2cc(CC=C(C)C)c(O)cc2O)ccc1O]

¹H NMR (400 MHz, acetone) δ 13.51 (1H, s), 9.49 (1H, brs), 8.25 (1H, brs), 7.93 (1H, s), 7.82 (1H, d, J=15.6 Hz), 7.76 (1H, d, J=15.6 Hz), 7.47 (1H, d, J=2.0 Hz), 7.33 (1H, dd, J=8.4, 2.0 Hz,), 6.93 (1H, d, J=8.0 Hz), 6.42 (1H, s), 5.38-5.33 (1H, m), 3.96 (3H, s), 3.30 (2H, d, J=7.2 Hz), 1.75 (6H, s); ¹³C NMR (100 MHz, acetone) δ 192.6, 165.9, 148.7, 145.2, 132.5, 132.1, 128.0, 124.5, 123.8, 121.2, 118.6, 116.2, 114.3, 112.1, 103.4, 56.3, 28.6, 25.8, 17.9; LRMS (ESI) calcd. for C₂₁H₂₃O₅ [M+H]⁺: 355.14, found: 355.10; HRMS (ESI) calcd. for C₂₁H₂₃O₅ [M+H]⁺: 355.1467, found: 355.1551; m.p=157.3° C.

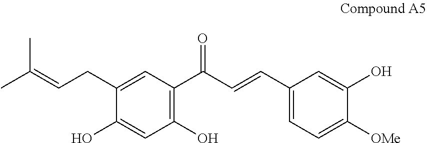 [SMILES: COc1ccc(/C=C/C(=O)c2cc(CC=C(C)C)c(O)cc2O)cc1O]

¹H NMR (400 MHz, acetone) δ 13.49 (1H, s), 9.49 (1H, brs), 8.01 (1H, brs), 7.81-7.72 (2H, m), 7.36 (1H, d, J=2.0 Hz), 7.26 (1H, dd, J=8.4, 2.0 Hz), 7.04 (1H, d, J=8.4 Hz,), 6.42 (s, 1H), 55.38-5.33 (1H, m), 3.93 (3H, s), 3.32 (2H, d, J=7.2 Hz), 1.74 (6H, d, J=8.0 Hz); ¹³C NMR (100 MHz, acetone) δ 192.6, 165.9, 165.4, 163.5, 150.9, 147.7, 144.9, 132.3, 129.2, 123.9, 123.4, 121.3, 119.3, 114.7, 114.3, 112.2, 103.4, 56.3, 28.8, 25.8, 17.9; LRMS (ESI) calcd. for C₂₁H₂₃O₅ [M+H]⁺: 355.14, found: 355.10; HRMS (ESI) calcd. for C₂₁H₂₃O₅ [M+H]⁺: 355.1467, found: 355.1549; m.p=159.1° C.

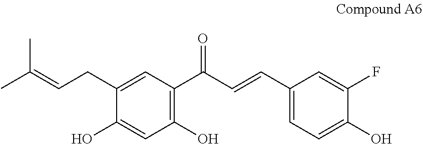 [SMILES: CC(C)=CCc1cc(C(=O)/C=C/c2ccc(O)c(F)c2)c(O)cc1O]

¹H NMR (400 MHz, acetone) δ 13.42 (1H, s), 9.39 (1H, brs), 8.00 (1H, s), 7.81 (2H, d, J=3.2 Hz), 7.68 (1H, dd, J=12.4, 2.0 Hz,), 7.49 (1H, dd, J=9.2, 2.8 Hz,), 7.09 (1H, t, J=8.8 Hz,), 6.42 (1H, s), 5.38-5.33 (1H, m), 3.31 (2H, d, J=7.2 Hz,), 1.74 (6H, d, J=9.2 Hz); ¹³C NMR (100 MHz, acetone) δ 192.5, 165.9, 165.5, 163.6, 152.4 (d, J=239.8 Hz), 148.2, 143.6, 132.3, 128.6, 127.4, 123.9, 121.4, 120.2, 118.9, 116.3 (d, J=18.6 Hz), 114.2, 103.4, 28.8, 25.8, 17.9; LRMS (ESI) calcd. for C₂₀H₂₀FO₄ [M+H]⁺: 343.12, found: 343.10; HRMS (ESI) calcd. for C₂₀H₂₀FO₄ [M+H]⁺: 355.1267, found: 343.1350; m.p=160.8° C.

### Preparation Example 2

A compound in which the substituent R₁ is H or halogen in Formula 1 was prepared according to the following steps.

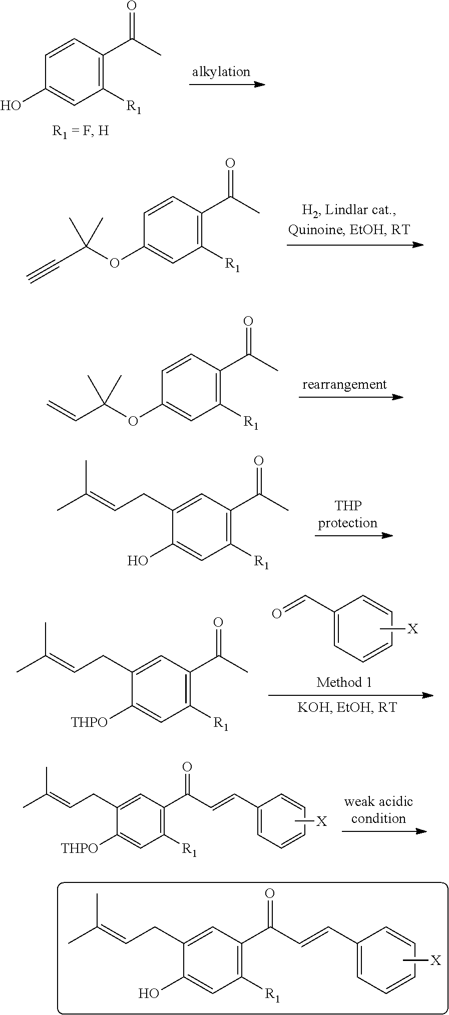

The triple bond of the intermediate was reduced to a double bond using a Lindlar catalyst, and a desired acetophenone was produced through a rearrangement reaction. Thereafter, a condensation reaction between acetophenone and various benzaldehydes was performed, and then the THP group was deprotected under weakly acidic conditions to obtain the desired final product.

Chemical structures and ¹H NMR, ¹³C NMR, LRMS, HRMS, and m.p values of Compounds B1 to B6 and C1 to C6 prepared in Preparation Example 2 are shown below, respectively.

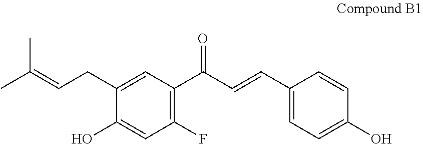 [SMILES: CC(C)=CCc1cc(C(=O)/C=C/c2ccc(O)cc2)c(F)cc1O]

¹H NMR (400 MHz, acetone) δ 7.69-7.60 (4H, m), 7.34 (1H, dd, J=15.6, 3.2 Hz,), 6.93 (2H, d, J=8.8 Hz,), 6.70 (1H, d, J=12.4 Hz,), 5.37-5.33 (1H, m), 3.35 (2H, d, J=7.2 Hz,), 1.74 (6H, s); ¹³C NMR (100 MHz, acetone) δ 186.9, 161.8 (d, J=248.6 Hz), 160.8, 160.7, 143.9, 133.2, 132.6, 131.2, 127.6, 125.8, 123.7, 122.9, 119.5 (d, J=12.6 Hz), 116.7, 103.2 (d, J=26.3 Hz), 28.2, 25.8, 17.8; LRMS (ESI) calcd. for C₂₀H₂₀FO₃ [M+H]⁺: 327.13, found: 327.10; HRMS (ESI) calcd. for C₂₀H₂₀FO₃ [M+H]⁺: 327.1318, found: 327.1402; m.p=145.3° C.

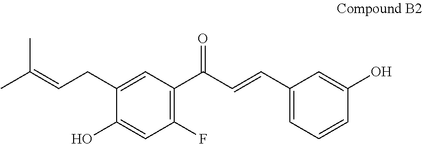 [SMILES: CC(C)=CCc1cc(C(=O)/C=C/c2cccc(O)c2)c(F)cc1O]

¹H NMR (400 MHz, acetone) δ 9.57 (1H, brs), 8.56 (1H, brs), 7.66-7.62 (2H, m), 7.45 (1H, dd, J=15.6, 3.2 Hz), 7.29 (1H, t, J=7.6 Hz), 7.22-7.18 (2H, m), 6.94 (1H, ddd, J=8.0, 2.8, 1.2 Hz,), 6.72 (1H, d, J=12.4 Hz), 5.38-5.33 (1H, m), 3.35 (2H, d, J=8.0 Hz), 1.75 (6H, s); ¹³C NMR (100 MHz, acetone) δ 186.9, 162.1 (d, J=249.0 Hz), 161.1, 158.7, 143.6, 137.4, 133.3, 132.6, 130.9, 126.6, 122.8, 120.8, 119.2 (d, J=12.4 Hz), 118.4, 115.3, 103.3 (d, J=26.3 Hz), 28.2, 25.8, 17.8; LRMS (ESI) calcd. for C₂₀H₂₀FO₃ [M+H]⁺: 327.13, found: 327.10; HRMS (ESI) calcd. for C₂₀H₂₀FO₃ [M+H]⁺: 327.1318, found: 327.1402; m.p=161.8° C.

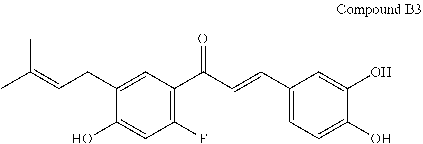 [SMILES: CC(C)=CCc1cc(C(=O)/C=C/c2ccc(O)c(O)c2)c(F)cc1O]

¹H NMR (400 MHz, acetone) δ 8.43 (1H, brs), 7.65 (1H, d, J=8.4 Hz), 7.60 (1H, dd, J=15.6, 2.0 Hz), 7.29 (1H, dd, J=15.6, 3.2 Hz), 7.24 (1H, d, J=2.4 Hz), 7.12 (1H, dd, J=8.4, 2.4 Hz), 6.90 (1H, d, J=8.0 Hz), 6.70 (1H, d, J=12.4 Hz), 5.37-5.32 (1H, m), 3.34 (2H, d, J=7.2 Hz), 1.75 (6H, s); ¹³C NMR (100 MHz, acetone) δ 186.9, 161.9 (d, J=248.4 Hz), 161.1, 160.8, 148.9, 145.3 (d, J=198.2 Hz), 133.2, 132.6, 128.2, 125.9, 123.7, 122.9, 120.8, 119.5 (d, J=12.7 Hz), 116.4, 115.3, 103.2 (d, J=26.5 Hz), 28.2, 25.8, 17.8; LRMS (ESI) calcd. for C₂₀H₂₀FO₄ [M+H]⁺: 343.12, found: 343.10; HRMS (ESI) calcd. for C₂₀H₂₀FO₄ [M+H]⁺: 343.1267, found: 343.1350; m.p=174.1° C.

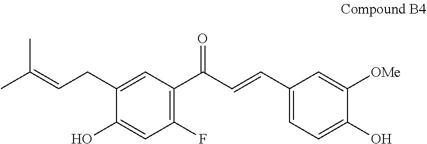 [SMILES: COc1cc(/C=C/C(=O)c2cc(CC=C(C)C)c(O)cc2F)ccc1O]

¹H NMR (400 MHz, acetone) δ 7.67-7.62 (2H, m), 7.37-7.32 (2H, m), 7.24 (1H, dd, J=8.0, 2.0 Hz), 6.91 (1H, d, J=8.0 Hz), 6.70 (1H, d, J=12.8 Hz), 5.37-5.33 (1H, m), 3.94 (3H, s), 3.34 (2H, d, J=7.2 Hz), 1.74 (6H, s); ¹³C NMR (100 MHz, acetone) δ 187.1, 161.8 (d, J=248.4 Hz), 160.7, 160.6, 150.2, 148.7, 144.4, 133.3, 132.5, 128.0, 125.8, 124.0, 122.8, 119.6 (d, J=12.7 Hz), 116.2, 111.9, 103.2 (d, J=26.2 Hz), 56.3, 28.2, 25.8, 17.8; LRMS (ESI) calcd. for C₂₁H₂₂FO₄ [M+H]⁺: 357.14, found: 357.10; HRMS (ESI) calcd. for C₂₁H₂₂FO₄ [M+H]⁺: 357.1424, found: 343.1510; m.p=172.2° C.

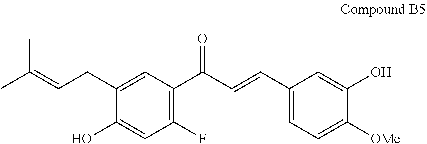 [SMILES: COc1ccc(/C=C/C(=O)c2cc(CC=C(C)C)c(O)cc2F)cc1O]

¹H NMR (400 MHz, acetone) δ 9.52 (1H, brs), 7.84 (1H, brs), 7.67-7.60 (2H, m), 7.34 (1H, dd, J=15.6, 2.8 Hz), 7.25 (1H, d, J=2.0 Hz), 7.19 (1H, dd, J=8.4, 2.0 Hz), 7.03 (1H, d, J=8.4 Hz), 6.71 (1H, d, J=12.4 Hz), 5.38-5.31 (1H, m), 3.92 (3H, s), 3.35 (2H, d, J=7.6 Hz), 1.74 (6H, s); NMR (100 MHz, acetone) δ 186.9, 161.9 (d, J=248.6 Hz), 150.7, 147.8, 143.9, 133.3, 132.6, 129.2, 125.8, 124.5, 124.4, 122.9, 122.8, 119.5 (d, J=12.6 Hz), 114.5, 112.3, 103.3 (d, J=26.5 Hz), 56.3, 28.2, 25.8, 17.8; LRMS (ESI) calcd. for C₂₁H₂₂FO₄ [M+H]⁺: 357.14, found: 357.10; HRMS (ESI) calcd. for C₂₁H₂₂FO₄ [M+H]⁺: 357.1424, found: 357.1509; m.p=167.0° C.

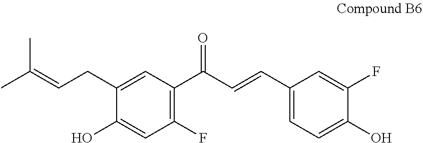 [SMILES: CC(C)=CCc1cc(C(=O)/C=C/c2ccc(O)c(F)c2)c(F)cc1O]

¹H NMR (400 MHz, acetone) δ 9.52 (1H, brs), 9.23 (1H, brs), 7.66-7.60 (2H, m), 7.56 (1H, dd, J=12.0, 2.0 Hz), 7.43 (1H, dd, J=8.4, 2.0 Hz), 7.38 (1H, dd, J=15.6, 2.8 Hz), 7.08 (1H, t, J=8.8 Hz), 6.71 (1H, d, J=12.4 Hz), 5.37-5.32 (1H, m), 3.35 (2H, d, J=7.6 Hz), 1.74 (6H, s); ¹³C NMR (100 MHz, acetone) δ 186.9, 161.9 (d, J=248.9 Hz), 161.0, 160.9, 152.4 (d, J=239.9 Hz), 148.0, 142.7, 133.3, 132.6, 128.5, 126.8, 125.9, 125.1 (d, J=7.9 Hz), 122.8, 119.4 (d, J=12.7 Hz), 118.9, 116.2 (d, J=18.5 Hz), 103.3 (d, J=26.4 Hz), 28.2, 25.8, 17.8; LRMS (ESI) calcd. for C₂₀H₁₉F₂O₃ [M+H]⁺: 345.12, found: 345.10; HRMS (ESI) calcd. for C₂₀H₁₉F₂O₃ [M+H]⁺: 345.1224, found: 345.1308; m.p=176.1° C.

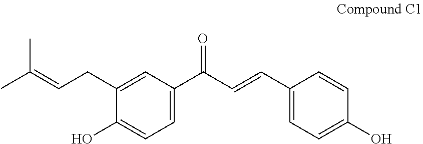 [SMILES: CC(C)=CCc1cc(C(=O)/C=C/c2ccc(O)cc2)ccc1O]

¹H NMR (400 MHz, acetone) δ 9.14 (1H, s), 8.92 (1H, s), 7.96 (1H, d, J=2.4 Hz), 7.92 (1H, dd, J=8.0, 2.0 Hz), 7.69 (4H, quar, J=2.4 Hz), 6.97 (1H, d, J=8.4 Hz), 6.93 (2H, d, J=8.4 Hz), 5.37-5.32 (1H, m), 3.35 (2H, d, J=7.6 Hz), 1.74 (6H, s); ¹³C NMR (100 MHz, acetone) δ 188.1, 160.5, 160.1, 143.7, 132.9, 131.5, 131.3, 131.2, 129.2, 129.1, 127.9, 123.2, 119.8, 116.7, 115.4, 29.1, 25.9, 17.9; LRMS (ESI) calcd. for C₂₀H₂₁O₃ [M+H]⁺: 309.14, found: 309.10; HRMS (ESI) calcd. for C₂₀H₂₁O₃ [M+H]⁺: 309.1412, found: 309.1497; m.p=75.1° C.

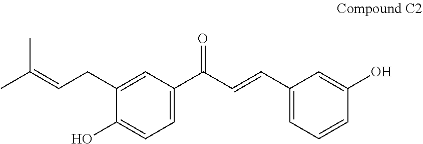 [SMILES: CC(C)=CCc1cc(C(=O)/C=C/c2cccc(O)c2)ccc1O]

¹H NMR (400 MHz, acetone) δ 9.19 (1H, brs), 8.59 (1H, brs), 7.96 (1H, d, J=2.4 Hz), 7.94 (1H, dd, J=8.4, 2.4 Hz), 7.77 (1H, d, J=15.6 Hz), 7.66 (1H, d, J=15.6 Hz), 7.27-7.24 (3H, m), 6.99 (1H, d, J=8.4 Hz), 6.95-6.92 (1H, m), 5.41-5.37 (1H, m), 3.41 (2H, d, J=8.0 Hz), 1.75 (6H, d, J=5.6 Hz); ¹³C NMR (100 MHz, acetone) δ 188.2, 160.3, 158.6, 143.6, 137.6, 132.9, 131.5, 131.2, 130.8, 129.4, 129.2, 123.2, 122.9, 120.7, 118.1, 115.6, 115.5, 29.1, 25.9, 17.9; LRMS (ESI) calcd. for C₂₀H₂₁O₃ [M+H]⁺: 309.14, found: 309.10; HRMS (ESI) calcd. for C₂₀H₂₁O₃ [M+H]⁺: 309.1412, found: 309.1495; m.p=200.9° C.

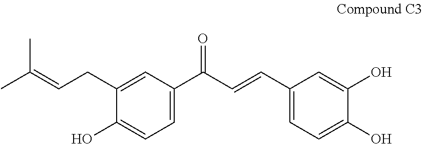 [SMILES: CC(C)=CCc1cc(C(=O)/C=C/c2ccc(O)c(O)c2)ccc1O]

¹H NMR (400 MHz, acetone) δ 9.12 (1H, brs), 7.95 (1H, d, J=2.4 Hz), 7.91 (1H, dd, J=8.4, 2.4 Hz), 7.62 (2H, d, J=4.0 Hz), 7.30 (1H, d, J=2.4 Hz), 7.17 (1H, dd, J=8.4, 2.4 Hz), 6.97 (1H, d, J=8.4 Hz), 6.90 (1H, d, J=8.0 Hz), 5.41-5.37 (1H, m), 3.41 (2H, d, J=7.6 Hz), 1.75 (6H, d, J=6.4 Hz); ¹³C NMR (100 MHz, acetone) δ 188.1, 160.1, 148.6, 146.2, 144.1, 132.9, 131.5, 131.3, 129.19, 129.12, 128.5, 123.2, 122.8, 119.9, 116.3, 115.5, 115.4, 29.1, 25.9, 17.9; LRMS (ESI) calcd. for C₂₀H₂₁O₄ [M+H]⁺: 325.13, found: 325.10; HRMS (ESI) calcd. for C₂₀H₂₁O₄ [M+H]⁺: 325.1362, found: 325.1444; m.p=92.1° C.

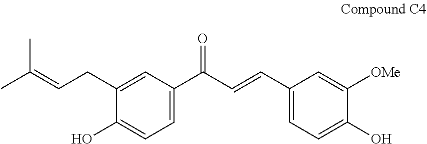 [SMILES: COc1cc(/C=C/C(=O)c2ccc(O)c(CC=C(C)C)c2)ccc1O]

¹H NMR (400 MHz, acetone) δ 9.14 (1H, brs), 8.19 (1H, brs), 7.95 (1H, d, J=2.4 Hz), 7.91 (1H, dd, J=8.4, 2.4 Hz), 7.70 (2H, s), 7.46 (1H, d, J=2.0 Hz), 7.28 (1H, dd, J=8.4, 2.0 Hz), 6.97 (1H, d, J=8.4 Hz), 6.91 (1H, d, J=8.0 Hz), 5.41-5.36 (1H, m), 3.94 (3H, s), 3.40 (2H, d, J=7.2 Hz), 1.75 (6H, d, J=6.5 Hz); ¹³C NMR (100 MHz, acetone) δ 188.1, 160.1, 150.0, 148.6, 144.2, 132.9, 131.5, 131.3, 129.2, 129.1, 128.3, 124.1, 123.2, 120.0, 116.1, 115.4, 111.7, 56.3, 29.0, 25.9, 17.9; LRMS (ESI) calcd. for C₂₁H₂₃O₄ [M+H]⁺: 339.15, found: 339.20; HRMS (ESI) calcd. for C₂₁H₂₃O₄ [M+H]⁺: 339.1518, found: 339.1601; m.p=64.2° C.

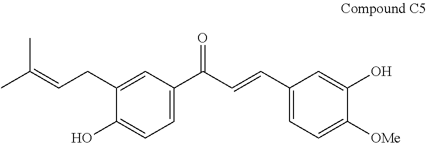 [SMILES: COc1ccc(/C=C/C(=O)c2ccc(O)c(CC=C(C)C)c2)cc1O]

¹H NMR (400 MHz, acetone) δ 7.94 (1H, d, J=2.4 Hz), 7.90 (1H, dd, J=8.4, 2.4 Hz), 7.68 (2H, s), 7.46 (1H, d, J=2.0 Hz), 7.28 (1H, dd, J=8.4, 2.0 Hz), 6.96 (1H, d, J=8.4 Hz), 6.91 (1H, d, J=8.0 Hz), 5.41-5.37 (1H, m), 3.95 (3H, s), 3.40 (2H, d, J=7.2 Hz), 1.75 (6H, d, J=4.8 Hz); ¹³C NMR (100 MHz, acetone) δ 188.1, 160.1, 150.5, 147.7, 143.8, 132.9, 131.5, 131.4, 129.5, 129.2, 129.1, 123.2, 122.9, 120.6, 115.5, 114.5, 112.2, 56.2, 29.1, 25.9, 17.9; LRMS (ESI) calcd. for C₂₁H₂₃O₄ [M+H]⁺: 339.15, found: 339.20; HRMS (ESI) calcd. for C₂₁H₂₃O₄ [M+H]⁺: 339.1518, found: 339.1601; m.p=147.3° C.

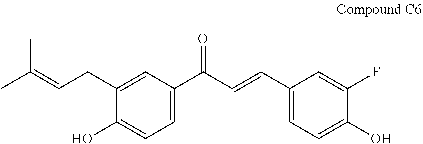 [SMILES: CC(C)=CCc1cc(C(=O)/C=C/c2ccc(O)c(F)c2)ccc1O]

¹H NMR (400 MHz, acetone) δ 9.34 (2H, brs), 7.97-7.93 (2H, m), 7.76-7.63 (3H, m), 7.46 (1H, dd, J=8.4, 1.6 Hz), 7.07 (1H, t, J=8.4 Hz), 6.98 (1H, d, J=8.4 Hz), 5.41-5.36 (1H, m), 3.40 (2H, d, J=7.2 Hz), 1.74 (6H, d, J=7.2 Hz); ¹³C NMR (100 MHz, acetone) δ 188.0, 160.3, 152.4 (d, J=239.7 Hz), 147.9, 147.8, 142.6, 132.9, 131.4, 131.2, 128.86, 128.80, 127.0, 123.2, 121.3, 118.8, 116.0 (d, J=18.6 Hz), 115.4, 29.1, 25.9, 17.9; LRMS (ESI) calcd. for C₂₀H₂₀FO₃ [M+H]⁺: 327.13, found: 327.10; HRMS (ESI) calcd. for C₂₀H₂₀FO₃ [M+H]⁺: 327.1318, found: 327.1404; m.p=87.6° C.

### Experimental Example 1

It is well known that the expression of inflammatory cytokines increases by LPS stimulation in RAW 264.7 macrophages and that nitric oxide (NO) increases by overexpression of iNOS protein. Excessive secretion of inflammatory cytokines and increase in NO are one of the causes of inflammatory bowel disease, and thus inhibiting or reducing them is used as one of the criteria for evaluating anti-inflammatory efficacy.

Particularly, as the most important pathological process causing mucosal damage in inflammatory bowel disease, the adhesion of monocytes activated by TNFα to the intestinal mucosa and migration into tissues has been recently reported. When TNFα is treated in colonic epithelial cells such as HT29, the expression of chemokines such as MCP-1 and IL-8 is increased, and the expression of adhesion molecules such as ICAM-1 and VCAM-1 is increased.

Accordingly, in Experimental Example 1, it was confirmed whether secretion of TNFα as an inflammatory cytokine can be inhibited by the Compound according to the present invention.

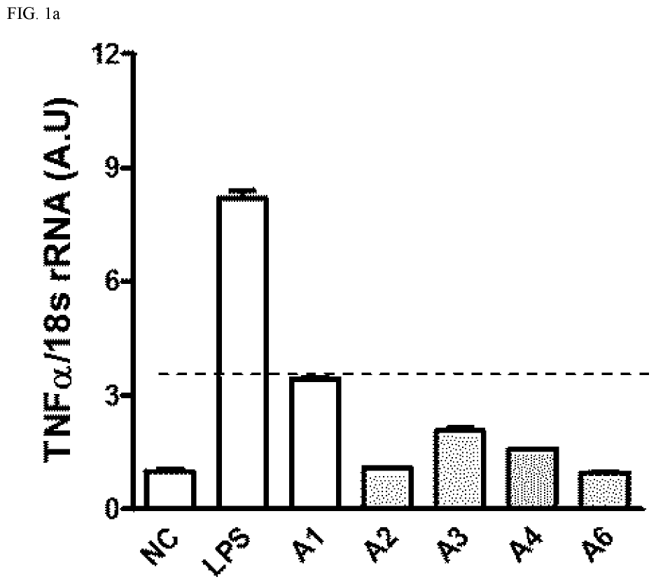

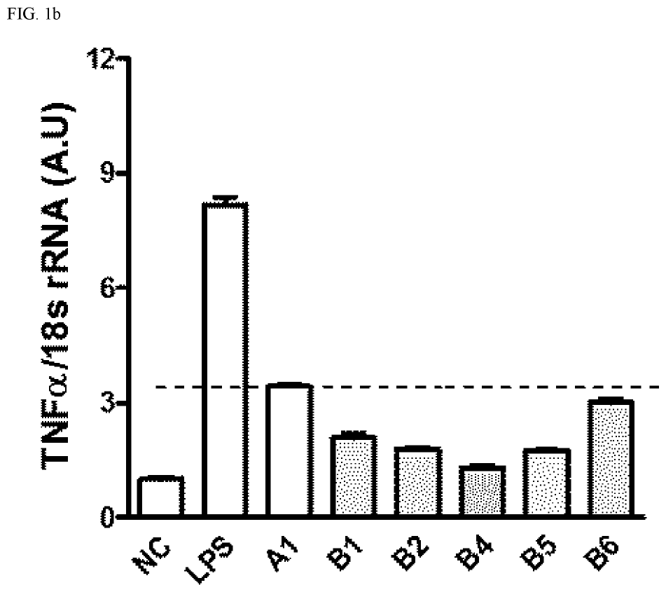

RAW 264.7 macrophages were treated with 0.1 μg/mL of E. coli-derived LPS and 25 μM of Compounds A1 to A6, B1 to B6, or C1 to C6, respectively. Then, the mRNA expression level of the inflammatory cytokine TNFα was observed through RT-PCR. The results are shown in FIGS. 1a to 1c.

As seen in FIGS. 1a to 1c, it shows that the mRNA expression level of TNFα in the E. coli-derived LPS treatment group was significantly increased compared to that in the NC group (negative control group).

The A1 treatment group shows that the increase in the mRNA expression level of TNFα induced by LPS stimulation is suppressed by treatment with broussochalcone A, which corresponds to the positive control group.

Groups treated with the compound according to the present invention show that the increase in the mRNA expression level of TNFα is significantly more suppressed than the A1 treatment group. This means that inflammatory cytokines, which are important factors in the mechanism of action of inflammatory bowel disease, can be significantly inhibited by the compound according to the present invention.

### Experimental Example 2

In Experimental Example 2, the mRNA expression level of inflammatory cytokines other than TNFα was observed in the same manner as in Experimental Example 1.

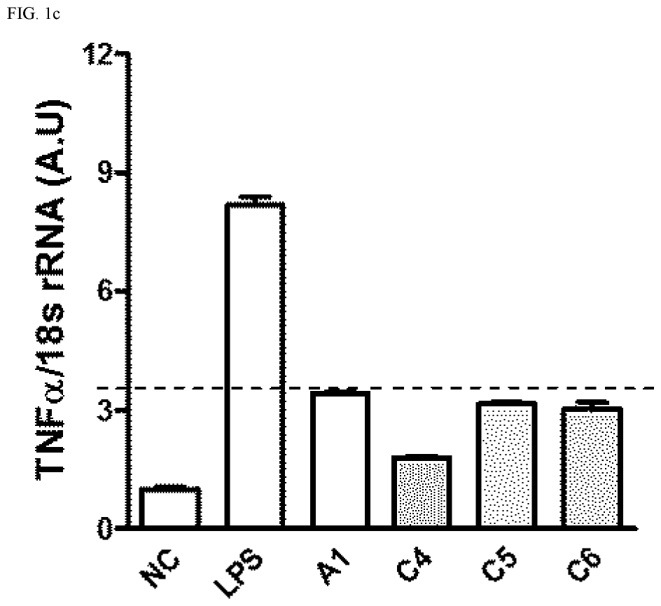

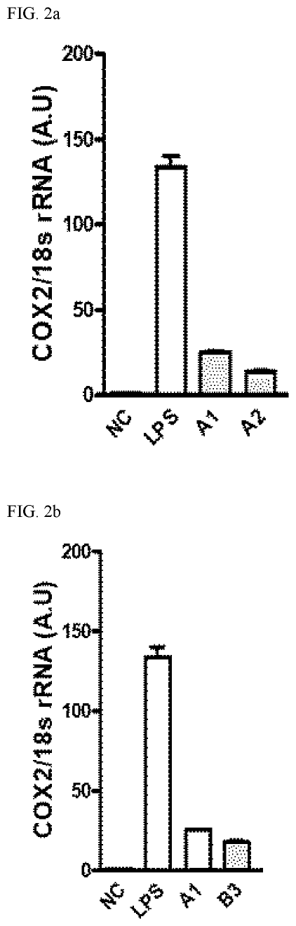

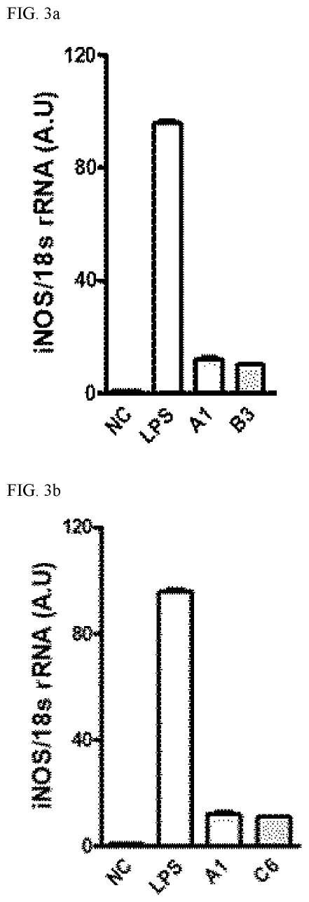

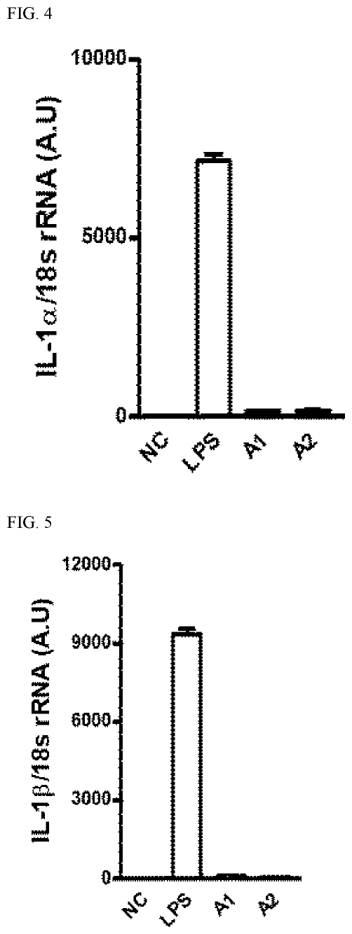

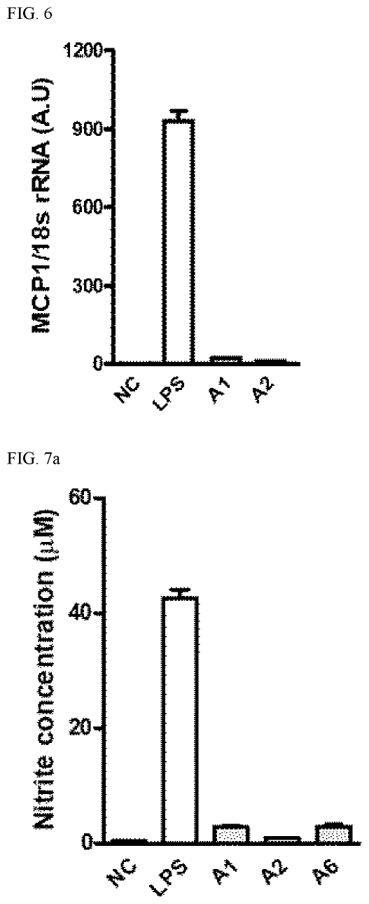

FIGS. 2a and 2b show the result of representing the mRNA expression level of COX2. FIGS. 3a and 3b show the result of representing the mRNA expression level of iNOS. FIG. 4 shows the result of representing the mRNA expression level of IL-1α. FIG. 5 shows the result of representing the mRNA expression level of IL-1β. FIG. 6 shows the result of representing the mRNA expression level of MCP1.

As seen in FIGS. 2 to 6, it shows that compared to the NC group (negative control group), the E. coli-derived LPS treatment group significantly increased the mRNA expression level of all other inflammatory cytokines like TNFα. In addition, it was also observed that the increase in the mRNA expression level of these inflammatory cytokines was suppressed by the treatment with broussochalcone A.

In the groups treated with the compound according to the present invention, the mRNA expression levels of COX2, iNOS, IL-1α, IL-1β, and MCP1 were significantly more suppressed than the A1 treatment group. This means that inflammatory cytokines, which are important factors in the mechanism of action of inflammatory bowel disease, can be significantly inhibited by the compound according to the present invention. Therefore, the compound according to the present invention is an active ingredient capable of treating, improving, and preventing inflammatory bowel disease which can exhibit more excellent effects than conventional therapeutic agents.

### Experimental Example 3

Nitric oxide (NO), which is increased by LPS stimulation in RAW 264.7 macrophages, is one of the causes of inflammatory bowel disease along with inflammatory cytokines. Therefore, in Experimental Example 3, it was confirmed in the same manner as in Experimental Example 1 whether NO produced by LPS stimulation could be reduced by the compound according to the present invention.

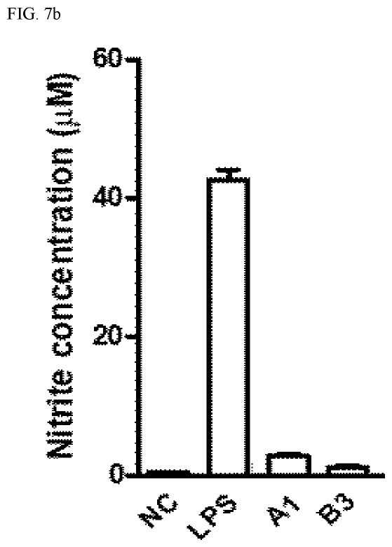

FIGS. 7a and 7b show the results of observing NO concentration (μM).

As seen in FIG. 7, compared to the NC group (negative control group), the E. coli-derived LPS treatment group significantly increased the NO level, which was suppressed by treatment with broussochalcone A. Groups treated with the compound according to the present invention significantly more suppressed the NO level than the A1 treatment group.

This means that nitric oxide, which is an important factor in the mechanism of action of inflammatory bowel disease, can be significantly inhibited by the compound according to the present invention. Therefore, the compound according to the present invention is an active ingredient capable of treating, improving, and preventing inflammatory bowel disease, and can exhibit more excellent effects than conventional therapeutic agents.

Therefore, the compound according to the present invention or a pharmaceutically or sitologically acceptable salt thereof can be usefully used as an active ingredient of a composition for treating, improving, or preventing inflammatory bowel disease.

1. A compound represented by Formula 1 or a pharmaceutically or sitologically acceptable salt thereof: 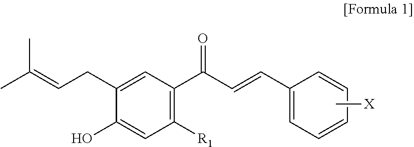
wherein,
R₁ is H, OH or halogen; and
X is one to five substituent group independently selected from H, OH, alkoxy and halogen,
provided that the compound is not broussochalcone A.

2. The compound or pharmaceutically or sitologically acceptable salt thereof according to claim 1, wherein X are two substituent groups.

3. The compound or pharmaceutically or sitologically acceptable salt thereof according to claim 1, wherein the compound is selected from the group consisting of:

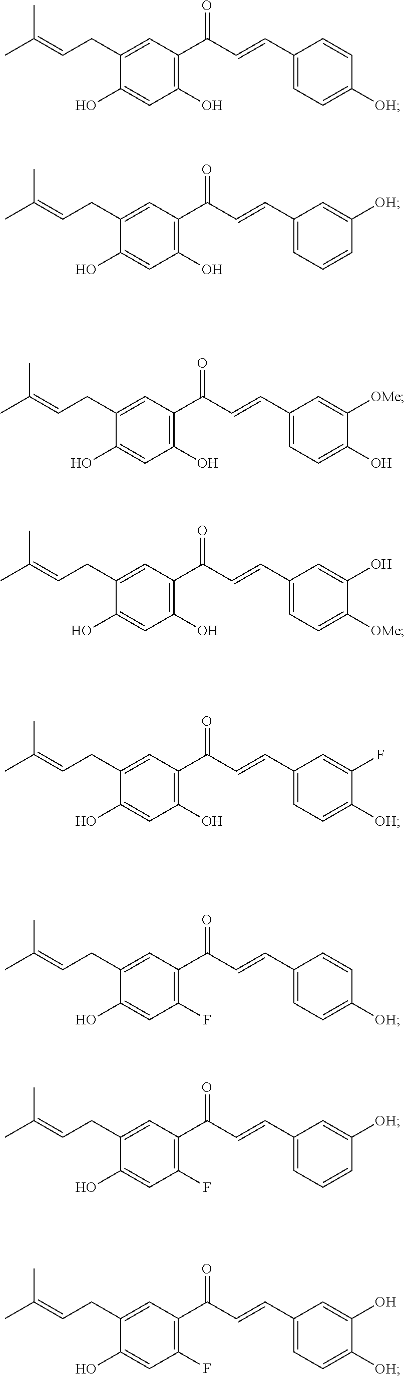

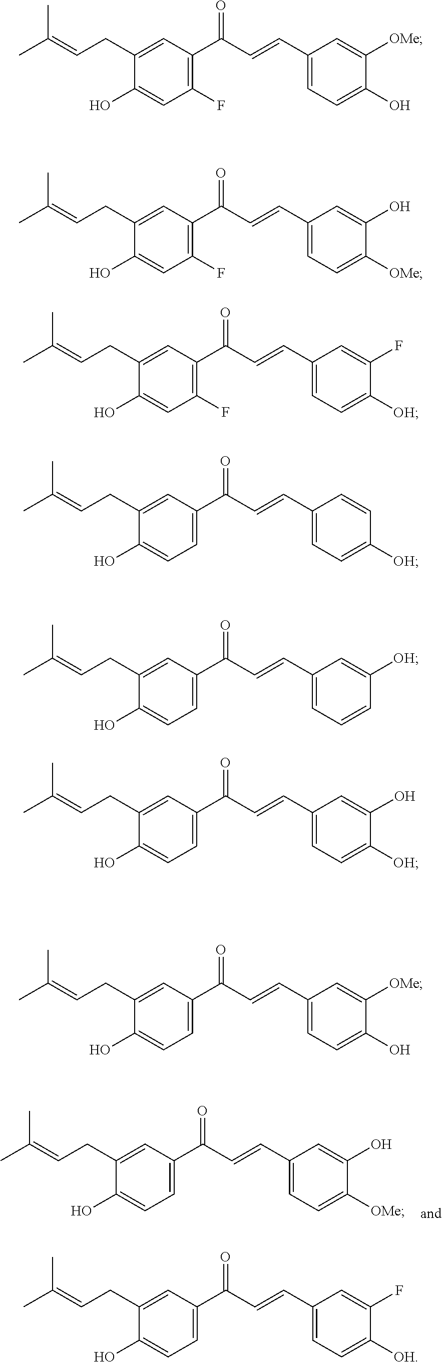

4. A pharmaceutical composition for treating or preventing an inflammatory bowel disease, comprising the compound or pharmaceutically acceptable salt thereof according claim 1.

5. The pharmaceutical composition according to claim 4, wherein the inflammatory bowel disease is selected from ulcerative colitis and Crohn's disease.

6. A food composition for improving or preventing an inflammatory bowel disease, comprising the compound or sitologically acceptable salt thereof according claim 1.

7. The food composition according to claim 6, wherein the inflammatory bowel disease is selected from ulcerative colitis and Crohn's disease.

8. A method for treating or preventing an inflammatory bowel disease, comprising administering the compound or pharmaceutically acceptable salt thereof according claim 1 to a subject in need thereof.
